(19) United States
(12) Patent Application Publication
(10) Pub. No.: US 2025/0213335 A1
(43) Pub. Date: Jul. 3, 2025

(54) ORAL CAVITY IMAGE PROCESSING DEVICE AND ORAL CAVITY IMAGE PROCESSING METHOD

(71) Applicant: MEDIT CORP, Seoul (KR)

(72) Inventors: Jung Kyen LEE, Seoul (KR); Seon Yae PARK, Seoul (KR)

(73) Assignee: MEDIT CORP., Seoul (KR)

(21) Appl. No.: 18/851,786

(22) PCT Filed: Mar. 30, 2023
(86) PCT No.: PCT/KR2023/004239

§ 371 Date: Sep. 27, 2024

(30) Foreign Application Priority Data
Mar. 30, 2022 (KR) 10-2022-0039905
Mar. 29, 2023 (KR) 10-2023-0041246

(51) Int. Cl. A61C 13/00; G06T 7/00; G06T 19/20

(52) U.S. Cl. CPC A61C 13/0004; G06T 7/0012; G06T 19/20; G06T 2200/04; G06T 2200/08; G06T 2200/24; G06T 2207/30036; G06T 2210/41

(57) ABSTRACT

Provided are an oral cavity image processing method and oral cavity image processing device. The oral cavity image processing method includes obtaining 3-dimensional oral cavity data by scanning an oral cavity including teeth, generating a base based on the 3-dimensional oral cavity data, generating one or more cylinders that pass through a wall of the base, based on a reference curve of the base, receiving a user input for moving a first cylinder from among the one or more cylinders, moving the first cylinder along the reference curve of the base based on the user input, and generating one or more drain holes by deleting data corresponding to the one or more cylinders from teeth model data including the base.

### TECHNICAL FIELD

Disclosed embodiments relate to an oral cavity image processing device and an oral cavity image processing method, and more particularly, to an oral cavity image processing device and method for generating a drain hole in teeth model data.

### BACKGROUND ART

There are various areas in dental treatment of patients. Examples of dental treatment areas may include orthodontics and prosthetic treatment. Teeth models of patients may be required for orthodontics and prosthetic treatment. Teeth models may be objective data showing the appearances before the start of orthodontics, during orthodontics, and after orthodontics. In addition, by making teeth models of portions that are difficult to directly observe due to limited spaces in oral cavities, direct observation is facilitated. In addition, spaces required to correct snaggleteeth, tooth protrusion, gaps between teeth may be accurately measured. In addition, when dentists counsel patients, teeth models may be objective data showing intraoral conditions of patients, thereby helping the understanding of patients. Furthermore, devices that are difficult to directly make in the oral cavities of patients may be made by using models.

Such teeth models are manufactured by 3-dimensional (3D) printers, and manufacturing processes by 3D printers require certain materials (resins). In many cases, to save materials (resins), teeth models are manufactured as hollow models. To manufacture teeth models as hollow models, drain holes for removing resins inside teeth models are required.

More specifically, when the bottom surfaces of hollow models are attached to build plates, drain holes are used to remove the pressure in the hollow models or discharge resins out of the hollow models. Alternatively, when teeth models are manufactured as hollow models, drain holes are used to save resin materials or printing time. Alternatively, drain holes are used to easily remove teeth models from build plates after printing processes of teeth models.

Therefore, to manufacture teeth models as hollow models, an operation of generating drain holes in teeth models is required.

### DISCLOSURE

### Technical Problem

Disclosed embodiments provide a method of processing an oral cavity image for generating drain holes in a teeth model and a device for performing operations according to the method.

### Technical Solution

According to an embodiment, an oral cavity image processing method may include obtaining 3-dimensional oral cavity data by scanning an oral cavity including teeth, generating a base based on the 3-dimensional oral cavity data, generating, based on a reference curve of the base, one or more cylinders that pass through a wall of the base, receiving a user input for moving a first cylinder from among the one or more cylinders, moving the first cylinder along the reference curve of the base, and generating one or more drain holes by deleting data corresponding to the one or more cylinders from teeth model data including the base.

According to an embodiment, the reference curve of the base may include at least one of an inner boundary of the base, an outer boundary of the base, and a curve connecting points located in a middle of the base in a thickness direction of the base.

According to an embodiment, a direction of a central axis of each of the one or more cylinders may be parallel to a direction of a normal vector of a point where the central axis intersects the reference curve.

According to an embodiment, the moving of the first cylinder along the reference curve of the base may include generating a virtual ray in a direction perpendicular to a screen from a first point on the screen, the first point corresponding to the user input, determining a second point on the reference curve, the second point being at a minimum distance from the virtual ray, and moving the first cylinder to locate the second point on the central axis of the first cylinder.

According to an embodiment, the determining of the second point may include determining the second point on the reference curve to be at a minimum distance from the virtual ray and within a preset distance from a third point on the reference curve, the third point being an intersection point between the reference curve and the central axis of the first cylinder before the first cylinder is moved.

According to an embodiment, the moving of the first cylinder along the reference curve of the base may include moving the first cylinder such that a distance between the central axis of the first cylinder and a reference surface of the base is maintained.

According to an embodiment, the oral cavity image processing method may further include displaying the teeth model data in which the one or more drain holes are generated.

According to an embodiment, the oral cavity image processing method may further include obtaining the teeth model data by generating mesh data extending from an edge of the 3-dimensional oral cavity data to the base.

According to an embodiment, the generating of the one or more cylinders may include receiving a user input regarding a number of the one or more cylinders, diameters of the one or more cylinders, and distances from a reference surface of the base to the one or more cylinders.

According to an embodiment, the generating of the one or more cylinders may further include determining positions of the one or more cylinders, based on the number of the one or more cylinders and the distances from the reference surface of the base to the one or more cylinders.

According to an embodiment, the generating of the one or more cylinders may include determining heights of side surfaces of the one or more cylinders such that the one or more cylinders pass through the wall of the base, and generating the one or more cylinders, based on the determined heights of the side surfaces of the one or more cylinders.

According to an embodiment, an oral cavity image processing device may include a display, a user interface, a memory storing one or more instructions, and a processor, wherein the processor is configured to execute the one or more instructions stored in the memory to obtain 3-dimensional oral cavity data by scanning an oral cavity including teeth, generate a base based on the 3-dimensional oral cavity data, generate, based on a reference curve of the base, one or more cylinders that pass through a wall of the base, control the display to display the generated one or more cylinders, receive, through the user interface, a user input for moving a first cylinder from among the one or more cylinders, move the first cylinder along the reference curve of the base based on the user input, and generate one or more drain holes by deleting data corresponding to the one or more cylinders from teeth model data including the base.

### Advantageous Effects

According to an oral cavity image processing device and an oral cavity image processing method, according to disclosed embodiments, drain holes may be generated such that the drain holes pass through a wall of a base. Therefore, when a teeth model is manufactured as a hollow model, a material (for example, a resin) inside the teeth model may be easily discharged out of the teeth model, thereby saving a resin material and reducing printing time.

According to an oral cavity image processing device and an oral cavity image processing method, according to disclosed embodiments, positions of drain holes automatically generated may be manually adjusted, and thus, an unintended result may be avoided when final teeth model data is generated.

According to an oral cavity image processing device and an oral cavity image processing method, according to disclosed embodiments, when positions of drain holes are manually adjusted, a user may easily and accurately adjust the positions of the drain holes by moving the drain holes along a reference curve of a base.

### DESCRIPTION OF DRAWINGS

The present disclosure may be easily understood from a combination of the following detailed description and the accompanying drawings, and the reference numerals represent structural elements.

FIG. 1 is a diagram illustrating an oral cavity image processing system according to a disclosed embodiment.

FIG. 2 is a diagram illustrating an operation of an oral cavity image processing device to obtain scan data to generate teeth model data, according to an embodiment.

FIGS. 3 and 4 are diagrams illustrating an operation of an oral cavity image processing device to generate a drain hole, according to an embodiment.

FIG. 5 is a diagram illustrating an operation of an oral cavity image processing device to determine a position of a drain hole, according to an embodiment.

FIGS. 6 to 8 are diagrams illustrating an operation of an oral cavity image processing device to move a drain hole, based on a user input, according to an embodiment.

FIGS. 9 to 12 are diagrams illustrating an operation in the case where information regarding a drain hole after an oral cavity image processing device adjusts a position of a cylinder, according to an embodiment.

FIG. 13 is a diagram illustrating an example in which an oral cavity image processing device generates a drain hole, according to an embodiment.

FIG. 14 is a diagram illustrating an operation of an oral cavity image processing device to generate teeth model data, according to an embodiment.

FIG. 15 is a flowchart illustrating an oral cavity image processing method according to an embodiment.

FIG. 16 is a block diagram illustrating an oral cavity image processing device according to an embodiment.

### MODE FOR INVENTION

The specification describes the principle of the present disclosure and discloses embodiments, to clarify the scope of the present disclosure and for those of ordinary skill in the art to implement the present disclosure. Disclosed embodiments may be implemented in various ways.

Throughout the specification, like components are denoted by like reference numerals. The specification does not describe all elements of embodiments, and general descriptions in the art or repeated descriptions between embodiments are omitted. The term “part or portion” used herein may be implemented by software or hardware, and depending on embodiments, a plurality of parts or portions may be implemented by one unit or element, or one part or portion may include a plurality of units or elements. Hereinafter, the principle and embodiments of the present disclosure will be described with reference to the accompanying drawings.

Herein, an image may include an image (hereinafter, referred to as an “oral cavity image”) that shows at least one tooth or an oral cavity including at least one tooth.

In addition, herein, an image may be a 2-dimensional image of an object, or a 3-dimensional model or a 3-dimensional image stereoscopically showing an object. Furthermore, herein, an image may refer to data required to 2-dimensionally or 3-dimensionally represent an object, for example, raw data obtained by at least one image sensor, or the like. Specifically, the raw data, which is data obtained to generate an oral cavity image, may be data (for example, 2-dimensional data) obtained by at least one image sensor of an intraoral scanner when the inside of the oral cavity of a patient, which is an object, is scanned by using the intraoral scanner.

Herein, an “object” may include a tooth, a gingiva, at least a portion of an oral cavity, and/or an artificial structure (for example, an orthodontic appliance, an implant, an artificial tooth, an orthodontic aid tool inserted in an oral cavity, or the like) that may be inserted in an oral cavity. Here, the orthodontic appliance may include at least one of a bracket, an attachment, an orthodontic screw, a lingual orthodontic appliance, and a removable orthodontic retainer.

Hereinafter, embodiments are described in detail with reference to the accompanying drawings.

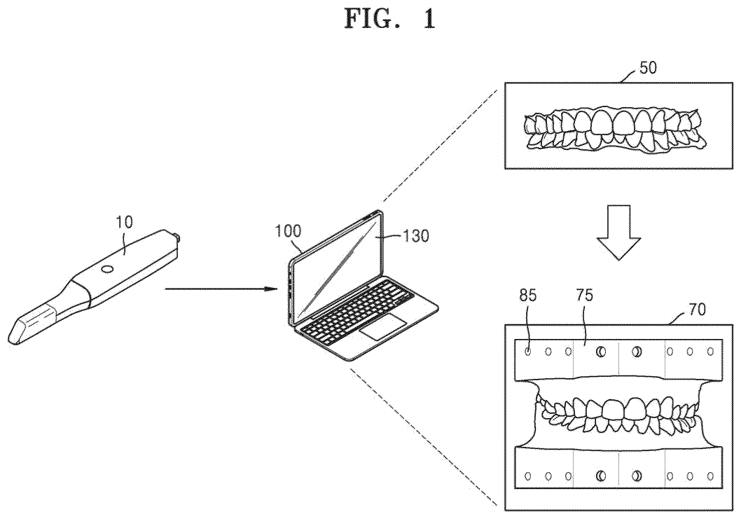

FIG. 1 is a diagram illustrating an oral cavity image processing system according to a disclosed embodiment.

Referring to FIG. 1, the oral cavity image processing system includes an intraoral scanner 10 and an oral cavity image processing device 100.

According to an embodiment, the intraoral scanner 10 is a device for scanning an object, that is, a medical device for obtaining an image of the inside of an oral cavity. In addition, the intraoral scanner 10 may scan at least a portion of a body of a patient, such as a face of a patient, or a teeth model, in addition to an oral cavity.

Although FIG. 1 illustrates that the intraoral scanner 10 is a type of a handheld scanner that a user holds in his hand to scan an object, the intraoral scanner 10 is not limited thereto and may include a scanner type, such as a model scanner that scans a mounted teeth model while moving around the teeth model.

Specifically, the intraoral scanner 10 may be a device for obtaining an image regarding an oral cavity including at least one tooth by being inserted into an oral cavity and then scanning teeth in a non-contact manner. In addition, the intraoral scanner 10 may be of a type capable of being put into and taking out of an oral cavity and scans the inside of the oral cavity of a patient by using at least one image sensor (for example, an optical camera or the like). The intraoral scanner 10 may obtain surface information of an object as raw data to image a surface of at least one of a tooth in an oral cavity that is an object, a gingiva in the oral cavity, and an artificial structure (for example, an orthodontic appliance including a bracket, a wire, and the like, an implant, an artificial tooth, an orthodontic aid tool inserted in an oral cavity, or the like) capable of being inserted into the oral cavity.

Image data obtained by the intraoral scanner 10 may be transmitted to the oral cavity image processing device 100 to which the intraoral scanner 10 is connected via a wired or wireless communication network.

The oral cavity image processing device 100 may include any electronic device connected to the intraoral scanner 10 via a wired or wireless communication network and capable of receiving a 2-dimensional image, which is obtained by scanning an oral cavity, from the intraoral scanner 10 and generating, processing, displaying, and/or transmitting an oral cavity image based on the received 2-dimensional image.

The oral cavity image processing device 100 may generate information by processing 2-dimensional image data or may generate an oral cavity image by processing the 2-dimensional image data, based on the 2-dimensional image data received from the intraoral scanner 10. In addition, the oral cavity image processing device 100 may display the generated information and the generated oral cavity image via the display 130.

The oral cavity image processing device 100 may include, but is not limited to, a computing device, such as a smartphone, a laptop computer, a desktop computer, a personal digital assistant (PDA), or a tablet personal computer (PC).

In addition, the oral cavity image processing device 100 may be present in the form of a server (or a server device) for processing the oral cavity image, or the like.

Furthermore, the intraoral scanner 10 may intactly transmit the raw data obtained by scanning the oral cavity to the oral cavity image processing device 100. In this case, the oral cavity image processing device 100 may generate a 3-dimensional oral cavity image 3-dimensionally representing the oral cavity, based on the received raw data. According to an embodiment, the oral cavity image processing device 100 may generate 3-dimensional data (for example, surface data, mesh data, or the like) 3-dimensionally representing the shape of a surface of an object, based on the received raw data.

In addition, because the 3-dimensional oral cavity image may be generated by 3-dimensionally modeling an inner structure of the oral cavity, based on the received raw data, the “3-dimensional oral cavity image” may be referred to as a “3-dimensional oral cavity model”. Hereinafter, a model or an image 2-dimensionally or 3-dimensionally representing an oral cavity are collectively referred to as an “oral cavity image”.

In addition, the oral cavity image processing device 100 may analyze, process, display, and/or transmit the generated oral cavity image to an external device.

As another example, the intraoral scanner 10 may obtain the raw data through oral cavity scanning, may generate an image corresponding to an oral cavity, which is an object, by processing the obtained raw data, and may transmit the generated image to the oral cavity image processing device 100. In this case, the oral cavity image processing device 100 may analyze, process, display, and/or transmit the received image.

In a disclosed embodiment, the oral cavity image processing device 100 includes an electronic device capable of generating and displaying an oral cavity image, which 3-dimensionally represents an oral cavity including one or more teeth, and the oral cavity image processing device 100 is described below in detail.

When the oral cavity image processing device 100 receives, from the intraoral scanner 10, the raw data obtained by scanning an oral cavity, the oral cavity image processing device 100 may generate a 3-dimensional oral cavity image (or a 3-dimensional oral cavity model) by processing the received raw data. For convenience of description, the 3-dimensional oral cavity image generated by the oral cavity image processing device 100 is referred to as “scan data” hereinafter.

The raw data received from the intraoral scanner 10 may include tooth data representing teeth and gingiva data representing gingivae. Therefore, the scan data generated by the oral cavity image processing device 100 may include a tooth region and a gingiva region. However, the present disclosure is not limited thereto.

According to an embodiment, when a teeth model is generated by using only scan data 50, a maxilla and a mandible, which are included in the scan data 50, are intactly separated, and thus, accurate occlusion of teeth may not be understood. Therefore, the teeth model needs to be generated in a shape to which an articulation for coupling the maxilla and the mandible to each other may be attached. To generate the teeth model in a shape to which an articulation may be attached, there is a need to generate teeth model data 70 combined with a base 75.

According to an embodiment, the oral cavity image processing device 100 may generate the teeth model data 70 combined with the base 75, by using the tooth region and a portion of the gingiva region of the scan data 50. Here, the oral cavity image processing device 100 may expand the gingiva region to the base 75 by generating 3-dimensional data (for example, mesh data) between an edge of the gingiva region and the base 75.

In addition, the teeth model may be manufactured as a hollow model in which the inside of the base 75 is emptied to use a less material. To manufacture the teeth model as a hollow model, there is a need for a drain hole 85 for discharging a material (for example, a resin) inside the teeth model. Here, to discharge the material through the drain hole 85, the drain hole 85 needs to be formed to pass through a wall of the base 75.

Hereinafter, an operation of the oral cavity image processing device 100 to generate the drain hole 85.

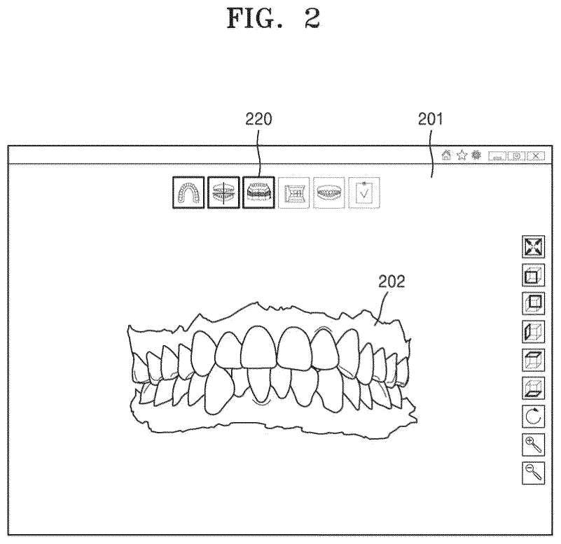

FIG. 2 is a diagram illustrating an operation of an oral cavity image processing device to obtain scan data to generate teeth model data, according to an embodiment.

According to an embodiment, the oral cavity image processing device 100 may generate scan data, based on the raw data obtained by the intraoral scanner 10. Alternatively, the oral cavity image processing device 100 may obtain pre-stored scan data by using a function of “load” or may obtain the scan data from an external device. However, the present disclosure is not limited thereto.

The oral cavity image processing device 100 may visually output scan data 202 via a user interface screen 201. The user interface screen 201 may correspond to a screen of the display 130 of FIG. 1. The user interface screen 201 may include at least one menu for allowing a user to analyze or process the scan data 202.

For example, the user interface screen 201 may include a base creation menu 220. When the oral cavity image processing device 100 receives a user input for selecting the base creation menu 220, the oral cavity image processing device 100 may enter a base creation mode.

According to an embodiment, the oral cavity image processing device 100 may generate a base corresponding to the scan data in the base creation mode and may provide menus capable of setting a base type, a base height, a hollow model or not, a wall thickness of a hollow model, the number of drain holes, and the like, which are required for base generation.

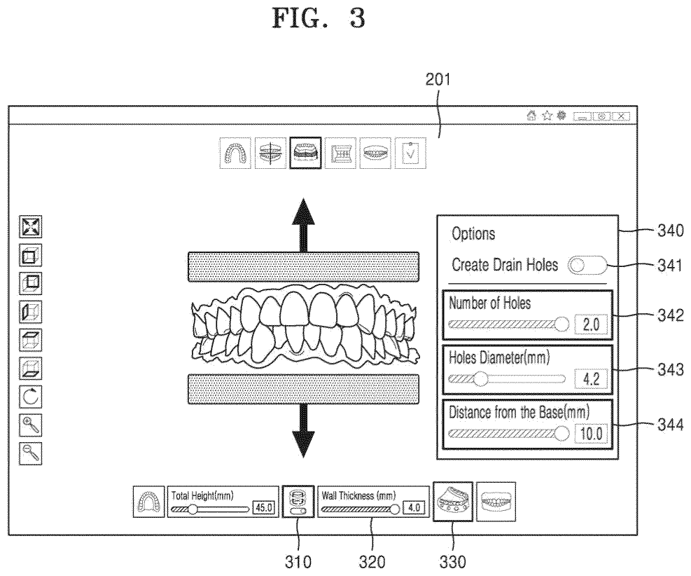

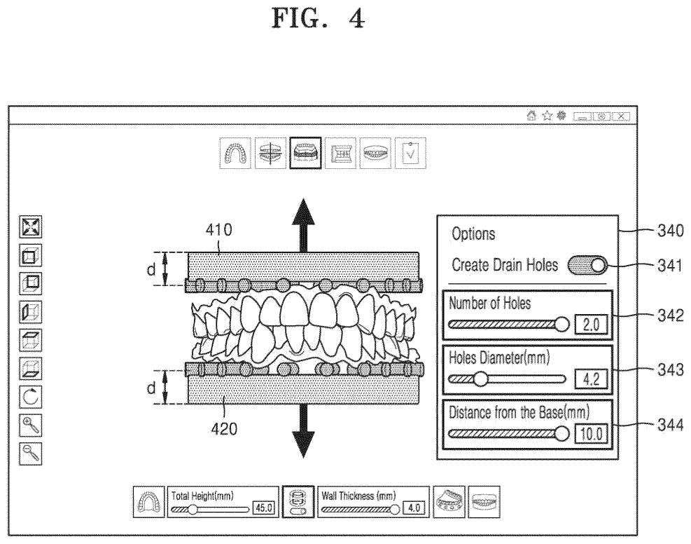

FIGS. 3 and 4 are diagrams illustrating an operation of an oral cavity image processing device to generate a drain hole, according to an embodiment.

Referring to FIG. 3, the oral cavity image processing device 100 according to an embodiment may display, on the user interface screen 201, a menu 310 capable of selecting a hollow model and a slider 320 for adjusting the thickness of the base wall, in the base creation mode. The oral cavity image processing device 100 may receive a user input for selecting the hollow model menu 310 and a user input for adjusting the thickness of the base wall by using the slider 320.

In addition, when a hollow model is selected, the oral cavity image processing device 100, based on the user input, may display a drain hole creation menu 330 on the user interface screen 201 or may activate the drain hole creation menu 330.

Furthermore, when the drain hole creation menu 330 is selected, the oral cavity image processing device 100 may display a drain hole setting menu 340 on the user interface screen 201, based on the user input, as shown in FIG. 3. Here, when a drain hole creation button 341 in the drain hole setting menu 340 is in an Off state, menus (that is, 342, 343, and 344) for setting information regarding drain holes may be deactivated.

When a user input for turning on the drain hole creation button 341 is received, the oral cavity image processing device 100 may activate menus (that is, 342, 343, and 344) for setting information regarding drain holes. The menus for setting information regarding drain holes may include drain hole setting menus capable of setting the number of drain holes, diameters of drain holes, and distances from the base to the drain holes (for example, distances from the bottom surface of the base to the drain holes).

For example, the drain hole creation button 341 may include a first slider 342 for adjusting the number of drain holes, a second slider 343 for adjusting the diameters of the drain holes, and a third slider 344 for adjusting vertical heights from a reference surface of the base (for example, the upper surface of the base or the lower surface of the base) to the drain holes. However, the present disclosure is not limited thereto.

When the information regarding drain holes is set through the drain hole setting menu 340, the oral cavity image processing device 100 may generate one or more cylinders respectively corresponding to one or more drain holes.

Here, the oral cavity image processing device 100 according to an embodiment may determine positions of the cylinders, based on the number of drain holes and the distances from the reference surface of the base to the drain holes.

The oral cavity image processing device 100 may determine vertical positions of the cylinders, based on the set distances from the reference surface of the base to the drain holes.

For example, when the distance from the reference surface of the base to each of the drain holes is set to d (for example, 10 mm), the oral cavity image processing device 100 may adjust the vertical positions of the cylinders such that the distance from the upper surface of a maxilla base 410 to a central axis of each of the cylinders is d (for example, 10 mm), and may adjust the vertical positions of the cylinders such that the distance from the lower surface of a mandible base 420 to the central axis of each of the cylinders is d (for example, 10 mm). Adjusting the vertical positions of the cylinders may be regarded as adjusting a vertical position of a reference curve of the base, the reference curve being described below.

Although it is illustrated and described with reference to FIG. 4 that the distance from the maxilla base 410 to each of the cylinders and the distance from the mandible base 420 to each of the cylinders are equally adjusted, the present disclosure is not limited thereto, and the distance from the maxilla base 410 to each of the cylinders and the distance from the mandible base 420 to each of the cylinders may be differently set.

In addition, the oral cavity image processing device 100 may determine horizontal positions of the cylinders, based on the number of drain holes and the reference curve of the base.

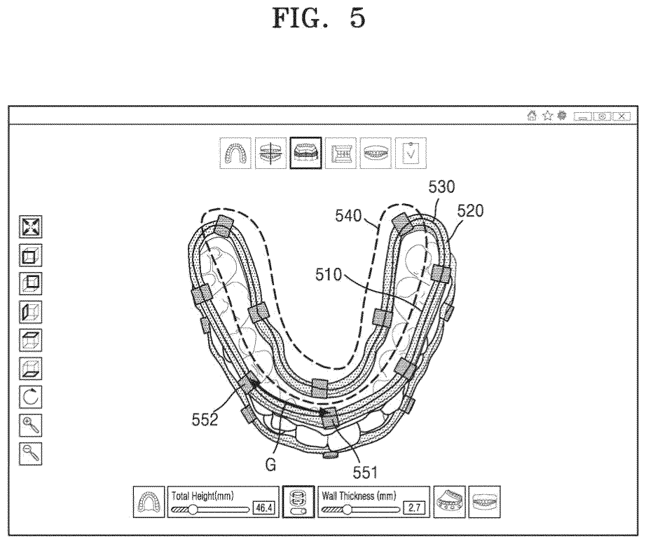

This is described in detail with reference to FIG. 5.

FIG. 5 is a diagram illustrating an operation of an oral cavity image processing device to determine a position of a drain hole, according to an embodiment.

Referring to FIG. 5, the oral cavity image processing device 100 may calculate a gap between the cylinders by dividing the length of the reference curve of the base by the set number of drain holes.

According to an embodiment, the reference curve of the base may be one of an inner boundary 510 of the base, an outer boundary 520 of the base, or a middle curve 530 connecting points located in the middle of the base in a thickness direction of the base. According to an embodiment, a vertical position of the reference curve of the base may be equal to the vertical position of each of the cylinders. Therefore, by adjusting the vertical position of each of the cylinders, the vertical position of the reference curve may also be adjusted. However, the present disclosure is not limited thereto.

For example, when the middle curve 530 of the base is taken as the reference curve of the base, the oral cavity image processing device 100 may calculate a gap G between the drain holes by dividing the length of the middle curve 530 of the base by the number of drain holes. The oral cavity image processing device 100 may determine a first point from among points of the middle curve 530 of the base as a position of a first cylinder 551 and may determine a second point separated from the first point by as much as the calculated gap G along the middle curve 530 of the base as a position of a second cylinder 552. The oral cavity image processing device 100 may determine positions of one or more cylinders in the same manner as described above. Therefore, when the number of drain holes increases, the gap between the cylinders decreases, and when the number of drain holes decreases, the gap between the cylinders increases.

In addition, the oral cavity image processing device 100 may determine the positions of the one or more cylinders by taking the inner boundary 510 of the base or the outer boundary 520 of the base as the reference curve of the base. However, the present disclosure is not limited thereto.

Alternatively, the oral cavity image processing device 100 may generate the cylinders corresponding to the drain holes only in a preset area. For example, when the drain holes are set to be generated only in a lingual-side area due to reasons such as engraving, the oral cavity image processing device 100 may generate the cylinders only in a base region 540 located on a lingual side of teeth. For example, by dividing the length of the reference curve (for example, one of the inner boundary, the outer boundary, and the middle curve) of the base in the base region 540 located on the lingual side by the number of drain holes, the gap between the drain holes may be obtained, and the cylinders corresponding to the drain holes may be generated only in the base region 540 corresponding to the lingual side.

In addition, the oral cavity image processing device 100 may determine a central axis direction of a cylinder, based on the reference curve of the base. For example, the oral cavity image processing device 100 may determine the central axis direction of the cylinder such that the central axis of the cylinder is parallel to a direction of a normal vector of a point on the reference curve of the base, the point being an intersection point between the reference curve of the base and the central axis of the cylinder. However, the present disclosure is not limited thereto.

Referring again to FIG. 4, the oral cavity image processing device 100 may determine the diameters of the lower surfaces of the cylinders according to the diameters of the drain holes. For example, when the diameter of each drain hole is set to 4.2 mm, the oral cavity image processing device 100 may generate the cylinders such that the diameter of the lower surface of each of the cylinders is 4.2 mm.

In addition, according to an embodiment, to discharge a material (a resin) inside the teeth model, the drain holes need to be formed to pass through the wall of the base. Therefore, the heights of the side surfaces of the cylinders corresponding to the drain holes need to be determined such that the cylinders pass through the wall of the base.

When the drain holes are automatically generated through the drain hole setting menus (that is, 342, 343, and 344), there may be a sharp portion at the base surface due to a position overlap between the drain holes, an articulation is not allowed to be attached due to the generation of a drain hole at an articulation position, or a drain hole may be generated at a label position.

Therefore, to adjust the positions of the drain holes that are automatically generated, there is a need to manually adjust the positions of the automatically generated drain holes through a user input.

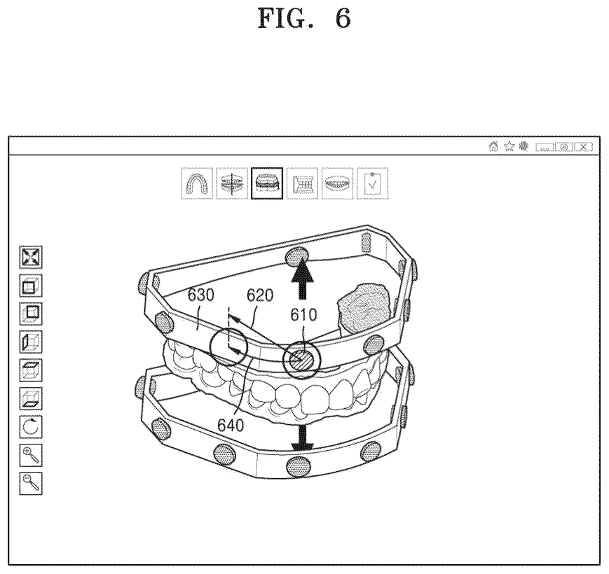

According to an embodiment, the oral cavity image processing device 100 may adjust the horizontal positions of the cylinders and the gap between the cylinders by an input for moving each cylinder by dragging each cylinder displayed on the user interface screen. This is described in detail with reference to FIGS. 6 to 8.

FIGS. 6 to 8 are diagrams illustrating an operation of an oral cavity image processing device to move a drain hole based on a user input, according to an embodiment.

Referring to FIG. 6, the oral cavity image processing device 100 according to an embodiment may generate one or more cylinders corresponding to one or more drain holes.

Because an operation of the oral cavity image processing device 100 to generate one or more cylinders has been described in detail with reference to FIGS. 3 to 5, repeated descriptions are omitted.

The oral cavity image processing device 100 may receive a user input for moving at least one of the one or more cylinders. For example, the oral cavity image processing device 100 may receive an input 620 for selecting a first cylinder 610 from among the one or more cylinders and dragging the first cylinder 610 by as much as a first distance in a first direction. However, the present disclosure is not limited thereto, and the oral cavity image processing device 100 may receive various types of user inputs for moving the first cylinder 610.

Here, the first cylinder 610 has a 3-dimensional coordinate value, and the user input for selecting the first cylinder 610 and dragging the first cylinder 610 by as much as the first distance in the first direction has a 2-dimensional coordinate value. For example, a pointer corresponding to the user input may move on a 2-dimensional screen. In addition, although the pointer corresponding to the user input may freely move on the screen, the first cylinder 610 needs to move on a base 630. Therefore, there is a need for a method of converting a movement corresponding to the user input, on 2-dimensional coordinates, into a movement on the base 630.

When the oral cavity image processing device 100 according to an embodiment receives the user input for selecting the first cylinder 610 and moving the first cylinder 610 on the 2-dimensional coordinates, the oral cavity image processing device 100 may move the first cylinder 610 on the base 630 by converting the user input having a 2-dimensional coordinate value into a movement on the base 630. For example, as shown in FIG. 6, when the oral cavity image processing device 100 receives a user input 620 for moving the first cylinder 610 by as much as the first distance in the first direction, the oral cavity image processing device 100 may move the first cylinder 610 by as much as a second distance 640 in a second direction along the reference curve of the base.

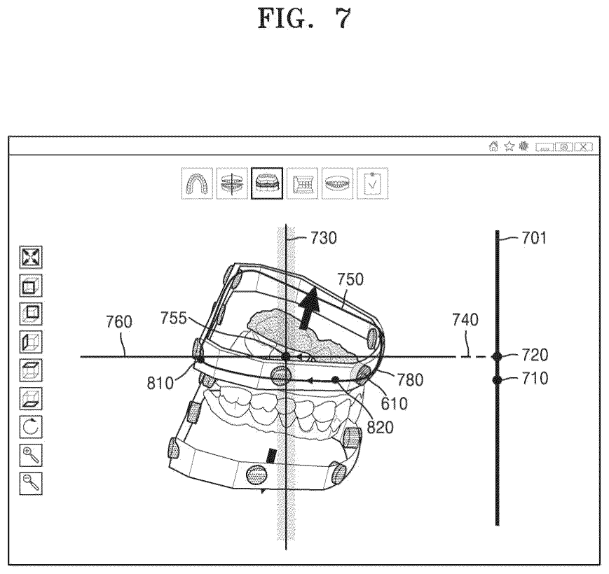

A method of converting a movement corresponding to a user input, on 2-dimensional coordinates, into a movement on a base, according to an embodiment, is described in detail with reference to FIG. 7.

FIG. 7 is a diagram illustrating a side view of a screen 701 according to an embodiment.

The oral cavity image processing device 100 according to an embodiment may receive an input for selecting and dragging the first cylinder 610, as shown in FIG. 6, and referring to FIG. 7, the user input 620 for selecting and dragging the first cylinder 610 may correspond to a movement from a first point 710 to a second point 720 on the screen 701.

When the oral cavity image processing device 100 according to an embodiment receives a user input, the oral cavity image processing device 100 may generate a virtual ray 740 heading toward a first plane 730, at a final position corresponding to the user input, for example, the second point 720. The first plane 730 may be a plane that is parallel to the screen 701 and passes through the center of a reference curve 750 of the base. However, the present disclosure is not limited thereto.

When the generated virtual ray 740 intersects the first plane 730 at a third point 755, the oral cavity image processing device 100 may generate a straight line 760 that passes through the third point 755 and is perpendicular to the screen 701 or the first plane 730. The oral cavity image processing device 100 may determine a movement position of the first cylinder 610, based on the distance between each of points on the reference curve 750 of the base and the generated straight line 760.

The oral cavity image processing device 100 according to an embodiment may determine, as the movement position of the first cylinder 610, a point that is on the reference curve of the base and has a minimum distance from the straight line 760.

However, when the movement position of the first cylinder 610 is determined by the method described above, there may be a situation not reflecting an intension of a user.

For example, as shown in FIG. 7, when scan data (for example, 3-dimensional oral cavity data) is inclined toward the screen 701, a fourth point 810 on the reference curve 750 of the base has a minimum distance from the straight line 760.

When the fourth point 810 is determined as the movement position of the first cylinder 610, the first cylinder 610 moves from the front side of the base to the back side of the base. In this case, the intention of the user to slightly move the position of the drain hole corresponding to the first cylinder 610 to the left may not be reflected. In general, when an input for selecting and dragging the first cylinder 610 is performed, this may be likely to be an intension to move the first cylinder 610 to an adjacent position.

Therefore, the oral cavity image processing device 100 according to an embodiment may determine the movement position of the first cylinder 610 in an area within a preset distance from the first cylinder 610. According to an embodiment, the oral cavity image processing device 100 may determine, as the movement position of the first cylinder 610, a point having a minimum distance from the straight line 760 from among points that are on the reference curve of the base and within a first threshold distance from the center of the first cylinder 610. For example, as shown in FIG. 7, the oral cavity image processing device 100 may determine that a fifth point 820 that is on a reference curve 780 of the base and within the first threshold distance from the center of the first cylinder 610 has a minimum distance from the straight line 760.

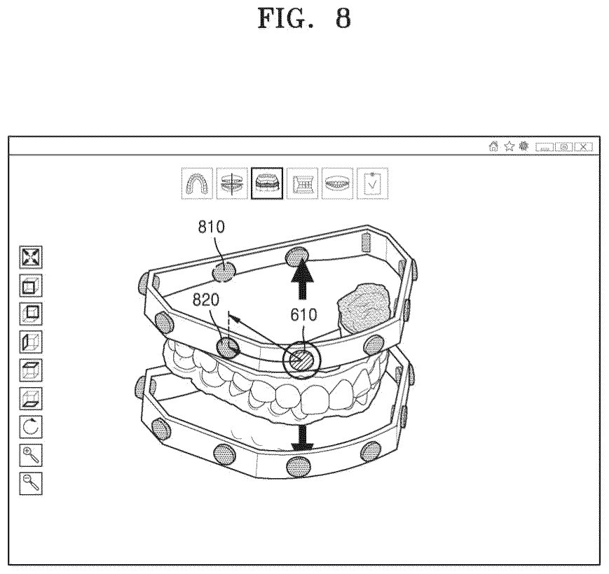

The oral cavity image processing device 100 may move the first cylinder 610 to the fifth point 820, as shown in FIG. 8.

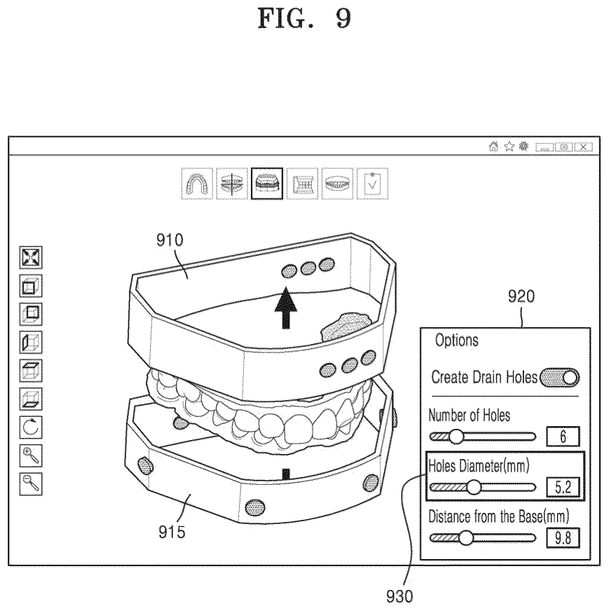

FIGS. 9 to 12 are diagrams illustrating an operation in the case where information regarding a drain hole is changed after an oral cavity image processing device adjusts a position of a cylinder, according to an embodiment.

As described with reference to FIGS. 6 to 8, the oral cavity image processing device 100 according to an embodiment may change the diameters and the vertical positions of the cylinders while the horizontal positions of the cylinders are maintained, after the horizontal position of at least one cylinder is changed based on a user input.

Referring to FIG. 9, at least one cylinder located in a maxilla base 910 may have a horizontal position that has been changed based on a user input. The oral cavity image processing device 100 may display a drain hole setting menu 920 on a user interface screen. The drain hole setting menu 920 may include menus for setting information regarding drain holes, and because this has been described in detail with reference to FIG. 3, repeated descriptions are omitted.

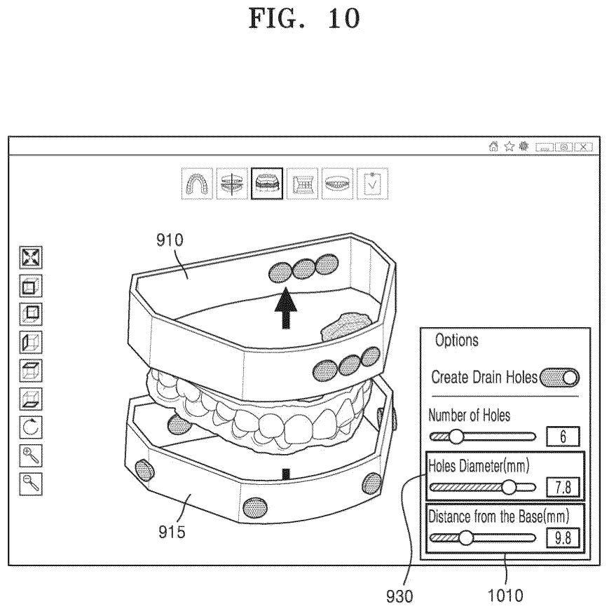

When a user input for changing the diameters of the drain holes is received through a menu 930 for adjusting the diameters of the drain holes, the oral cavity image processing device 100 may change the diameters of the cylinders while horizontal positions of cylinders located in the maxilla base 910 and horizontal positions of cylinders located in a mandible base 915 are maintained. For example, as shown in FIG. 10, when an input for moving a slider for adjusting the diameters of the drain holes to the right is received, the oral cavity image processing device 100 may increase the diameters of the cylinders while the horizontal positions of the cylinders located in the maxilla base 910 and the horizontal positions of the cylinders located in the mandible base 915 are maintained.

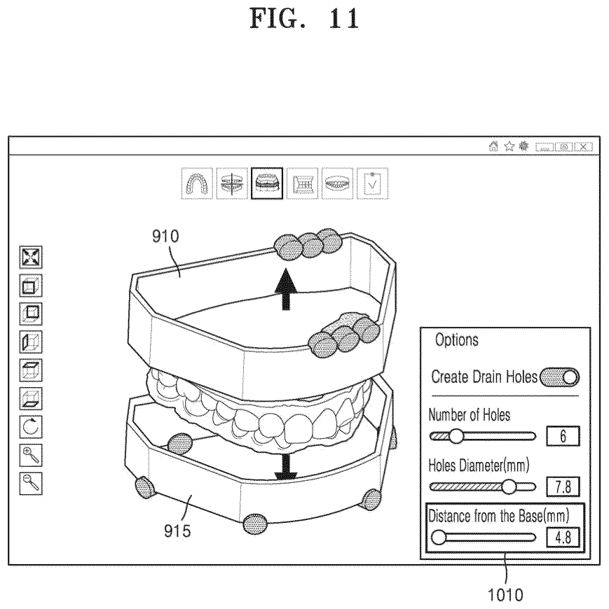

In addition, when a user input for changing the vertical positions of the drain holes is received through a menu 1010 for adjusting the distance from the base to each drain hole (for example, the vertical position of each drain hole), the oral cavity image processing device 100 may change the vertical positions of the cylinders while the horizontal positions of the cylinders located in the maxilla base 910 and the horizontal positions of the cylinders located in the mandible base 915 are maintained. For example, as shown in FIG. 11, when an input for moving a slider for adjusting the vertical height from the reference surface of the base to each drain hole to the left is received, the oral cavity image processing device 100 may moves the vertical positions of the cylinders such that the cylinders on the maxilla base 910 become closer to the upper surface of the maxilla base 910 and the cylinders on the mandible base become closer to the lower surface of the mandible base 915 while the horizontal positions of the cylinders located in the maxilla base 910 and the horizontal positions of the cylinders located in the mandible base 915 are maintained.

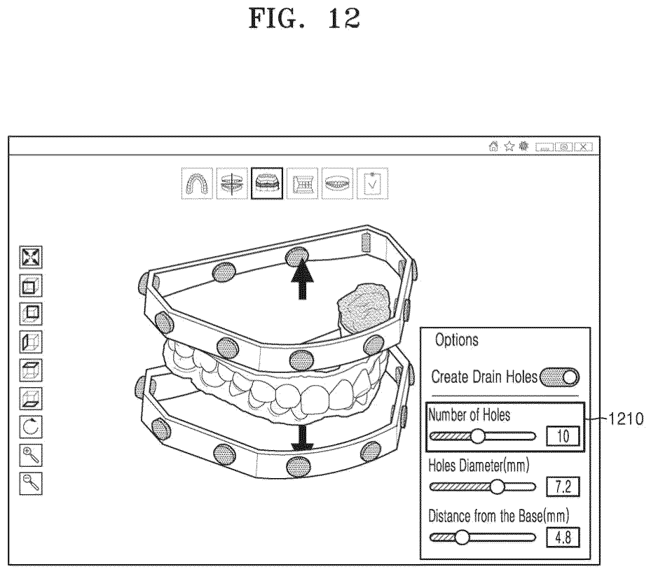

Referring to FIG. 12, the oral cavity image processing device 100 according to an embodiment may receive a user input for changing the number of drain holes through a menu 1210 for setting the number of drain holes.

When the oral cavity image processing device 100 according to an embodiment receives the user input for changing the number of drain holes, the manually adjusted positions of the cylinders may not be maintained. The oral cavity image processing device 100 may newly generate cylinders based on the changed number of drain holes. For example, cylinders corresponding to the drain holes may be generated by the automatic generation method described with reference to FIGS. 3 to 5.

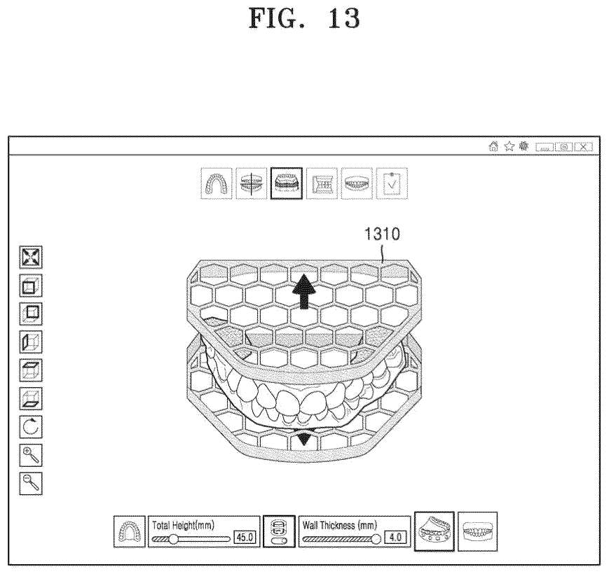

FIG. 13 is a diagram illustrating an example in which an oral cavity image processing device generates a drain hole, according to an embodiment.

Referring to FIG. 13, a base 1310 according to an embodiment may include a honeycomb structure. In addition, the base 1310 according to an embodiment may include a honeycomb structure while having a hollow shape. For example, the honeycomb structure refers to a honeycomb-shaped structure in which hexagons are connected to each other. The honeycomb structure is a stable structure distributing force in a balanced manner, and when a base according to an embodiment is generated in a honeycomb structure, the shape of the base may be prevented from being deformed.

According to an embodiment, the oral cavity image processing device 100 may also generate one or more drain holes in the base 1310 including a honeycomb structure, based on a reference curve of the base. For example, the oral cavity image processing device 100 may generate, in the base 1310, one or more cylinders respectively corresponding to the one or more drain holes, by the same method as the method described with reference to FIGS. 3 to 5.

In addition, the oral cavity image processing device 100 according to an embodiment may generate one or more drain holes in a wall of the honeycomb structure. Here, the number of drain holes and the sizes of the drain holes may be set based on the height of the wall of the honeycomb structure (for example, the height of the base). However, the present disclosure is not limited thereto.

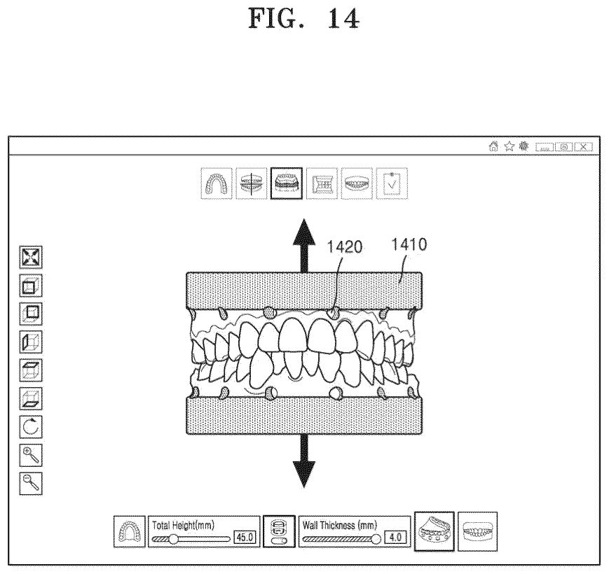

FIG. 14 is a diagram illustrating an operation of an oral cavity image processing device to generate teeth model data, according to an embodiment.

Referring to FIG. 14, the oral cavity image processing device 100 according to an embodiment may generate teeth model data combined with a base, by using scan data. The oral cavity image processing device 100 may generate mesh data between a base 1410 and an edge of a gingiva region selected from the scan data, thereby expanding the gingiva region to the base 1410. The base 1410 according to an embodiment may be generated as a hollow model, and drain holes 1420 may be generated in the teeth model data.

Drain holes according to an embodiment need to be generated to pass through a wall of a base to discharge a material (a resin) inside a teeth model. Therefore, the heights of side surfaces of cylinders, which respectively correspond to the drain holes according to an embodiment, may be determined such that the cylinders pass through the wall of the base. The oral cavity image processing device 100 may generate the drain holes 1420 by deleting, from the teeth model data, data corresponding to one or more cylinders generated to pass through the wall of the base 1410.

According to an embodiment, the oral cavity image processing device 100 may control the drain holes 1420 not to be generated in the gingiva region. For example, when the drain holes 1420 are set not to be generated in the gingiva region, a tooth region and the gingiva region, in the scan data, may be distinguished from each other. The oral cavity image processing device 100 may segment the tooth region and the gingiva region from the scan data. The segmenting of the tooth region and the gingiva region from the scan data may refer to separating teeth in the scan data from the gingiva region. The oral cavity image processing device 100 may segment the tooth region and the gingiva region by identifying scan data for teeth and scan data for gingivae, which are included in the scan data.

Alternatively, the oral cavity image processing device 100 may segment the tooth region and the gingiva region from the scan data by using artificial intelligence (AI) without an input by a user.

Alternatively, the oral cavity image processing device 100 may segment the tooth region and the gingiva region by using curvature information of the scan data.

When one or more cylinders overlap the tooth region, the oral cavity image processing device 100 may not delete data corresponding to the overlapping cylinders.

Alternatively, when one or more cylinders overlap the tooth region, the oral cavity image processing device 100 may adjust the positions of the overlapping cylinders, thereby placing the cylinders in the gingiva region or the base not to overlap the tooth region.

As shown in FIG. 14, after the drain holes 1420 are generated to pass through the wall of the base 1410 that is included in the teeth model data, when a teeth model of a hollow type is manufactured by using the teeth model data, a material (for example, a resin) inside the teeth model may be easily discharged through the drain holes 1420.

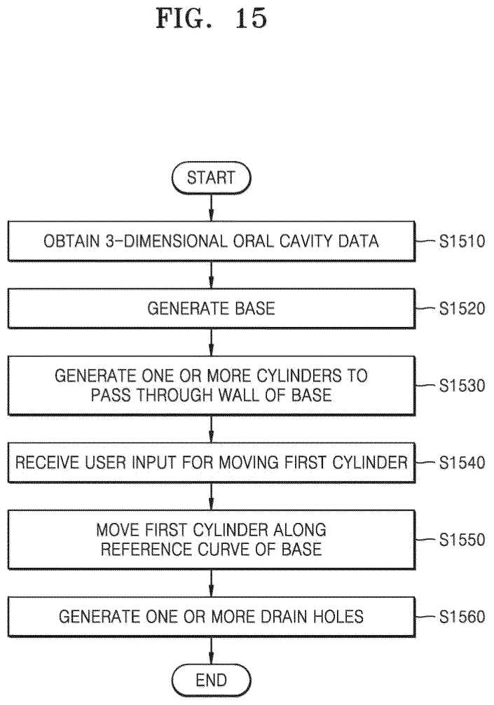

FIG. 15 is a flowchart illustrating an oral cavity image processing method according to an embodiment.

The oral cavity image processing method shown in FIG. 15 may be performed by the oral cavity image processing device 100.

Referring to FIG. 15, the oral cavity image processing device 100 according to an embodiment may obtain 3-dimensional oral cavity data (scan data) (S1510).

The oral cavity image processing device 100 may receive raw data, which is obtained by scanning the inside of an oral cavity including teeth or scanning a teeth model by using the intraoral scanner 10, and may generate scan data based on the received raw data. Alternatively, the oral cavity image processing device 100 may obtain the scan data stored in memory. Alternatively, the oral cavity image processing device 100 may obtain the scan data from an external device. However, the present disclosure is not limited thereto.

The oral cavity image processing device 100 may generate a base based on the scan data (S1520).

For example, the oral cavity image processing device 100 may enter a base creation mode by receiving a user input for selecting a base creation menu. The oral cavity image processing device 100 may generate a base corresponding to the scan data in the base creation mode and may provide menus capable of setting a base type, a base height, a hollow model or not, a wall thickness of a hollow model, the number of drain holes, and the like, which are required to generate the base.

The oral cavity image processing device 100 may generate one or more cylinders such that the one or more cylinders pass through the base wall, based on the base (S1530).

For example, when a menu for generating a base in a hollow shape is selected, the oral cavity image processing device 100 may provide or activate a drain hole creation menu. When an input for drain hole generation is received, the oral cavity image processing device 100 may provide menus for setting information regarding drain holes.

When the information regarding drain holes, including the number of drain holes, the diameters of the drain holes, the distances from the base to the drain holes, and the like, is set through a drain hole setting menu, the oral cavity image processing device 100 may generate cylinders corresponding to one or more drain holes.

Here, the oral cavity image processing device 100 may determine positions of the cylinders, based on the number of drain holes and the distances from the base to the drain holes. In addition, the oral cavity image processing device 100 may determine the heights of side surfaces of the cylinders such that the one or more cylinders pass through the wall of the base.

According to an embodiment, the oral cavity image processing device 100 may receive a user input for moving a first cylinder from among the generated one or more cylinders (S1540).

For example, the oral cavity image processing device 100 may receive an input for selecting the first cylinder from among the one or more cylinders and dragging the first cylinder by as much as a first distance in a first direction. However, the present disclosure is not limited thereto.

The oral cavity image processing device 100 according to an embodiment may move the first cylinder along a reference curve of the base, based on the user input (S1550).

According to an embodiment, the first cylinder has a 3-dimensional coordinate value, and the user input has a 2-dimensional coordinate value. For example, a pointer corresponding to the user input may move on a 2-dimensional screen. In addition, although the pointer corresponding to the user input may freely move on the screen, the first cylinder needs to move on the base. Therefore, there is a need for a method of converting a movement corresponding to the user input, on 2-dimensional coordinates, into a movement on the base.

Therefore, when a user input for selecting and moving the first cylinder is received, the oral cavity image processing device 100 may move the first cylinder on the base by converting the user input having a 2-dimensional coordinate value into a movement on the base. For example, when a user input for moving the first cylinder by as much as the first distance in the first direction, on 2-dimensional coordinates, is received, the oral cavity image processing device 100 may move the first cylinder by as much as a second distance in a second direction along the reference curve of the base.

Because the method of converting a movement corresponding to the user input, on 2-dimensional coordinates, into a movement on the base, according to an embodiment, has been described in detail with reference to FIG. 7, repeated descriptions are omitted.

The oral cavity image processing device 100 may generate one or more drain holes by deleting data corresponding to the one or more cylinders from the teeth model data (S1560).

Here, the oral cavity image processing device 100 may distinguish between a tooth region and a gingiva region in the scan data and may control the drain holes not to be generated in the tooth region. For example, when one or more cylinders overlap the tooth region, the oral cavity image processing device 100 may not delete data corresponding to the overlapping cylinders. Alternatively, when one or more cylinders overlap the tooth region, the oral cavity image processing device 100 may adjust positions of the overlapping cylinders and thus place the cylinders in the gingiva region or the base not to overlap the tooth region.

According to an embodiment, the oral cavity image processing device 100 may display the teeth model data in which the drain holes are generated.

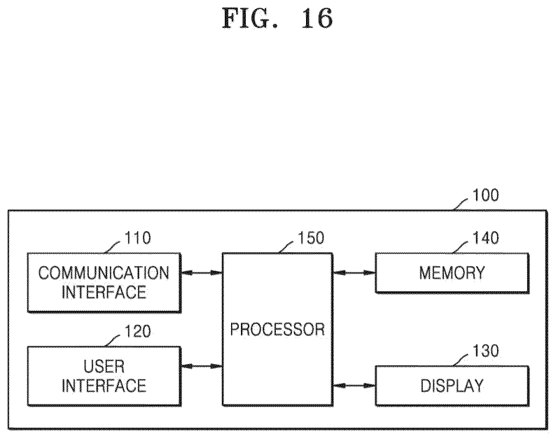

FIG. 16 is a block diagram illustrating an oral cavity image processing device according to an embodiment.

The oral cavity image processing method shown in FIG. 15 may be performed by the oral cavity image processing device 100. Therefore, the oral cavity image processing method shown in FIG. 15 may correspond to a flowchart illustrating operations of the oral cavity image processing device 100.

Referring to FIG. 16, the oral cavity image processing device 100 may include a communication interface 110, a user interface 120, a display 130, a memory 140, and a processor 150.

The communication interface 110 may communicate with at least one external electronic device (for example, the intraoral scanner 10, a server, an external medical device, or the like) through a wired or wireless communication network. The communication interface 110 may communicate with at least one external electronic device according to control by the processor 150.

Specifically, the communication interface 110 may include at least one short-range communication module for performing communication according to communication specifications, such as Bluetooth, WiFi, Bluetooth Low Energy (BLE), Near Field Communication (NFC)/Radio Frequency Identification (RFID), WiFi Direct, Ultra-Wideband (UWB), or ZIGBEE.

In addition, the communication interface 110 may further include a long-range communication module for communicating with a server for supporting long-range communication according to long-range communication specifications. Specifically, the communication interface 110 may include a long-range communication module for performing communication through a network for Internet communication. In addition, the communication interface 110 may include a long-range communication module for performing communication through a communication network conforming to communication specifications, such as 3G, 4G, and/or 5G.

Furthermore, to communicate with an external electronic device (for example, an intraoral scanner or the like) in a wired manner, the communication interface 110 may include at least one port for connection to the external electronic device by a wired cable. Therefore, the communication interface 110 may communicate with the external electronic device connected thereto in a wired manner, through the at least one port.

The user interface 120 may receive a user input for controlling the oral cavity image processing device 100. The user interface 120 may include, but is not limited to, a user input device including a touch panel for sensing a touch by a user, a button for receiving a push operation of a user, a mouse or a keyboard for specifying or selecting one point on a user interface screen, or the like. For example, the user interface 120 may receive a user input for setting options required to generate one or more drain holes. In addition, the user interface 120 may receive a user input for moving at least one of one or more cylinders generated on a base.

Furthermore, the user interface 120 may include a speech recognition device for speech recognition. For example, the speech recognition device may include a microphone and may receive a speech command or a speech request of a user. Therefore, the processor 150 may control an operation corresponding to the speech command or the speech request to be performed.

The display 130 displays a screen. Specifically, the display 130 may display a certain screen according to control by the processor 150. Specifically, the display 130 may display a user interface screen including an oral cavity image generated based on data that is obtained by scanning, by the intraoral scanner 10, an oral cavity of a patient. Alternatively, the display 130 may display a user interface screen including information related to the dental treatment of a patient.

The memory 140 may store at least one instruction. In addition, the memory 140 may have stored at least one instruction that is to be executed by the processor 150. In addition, the memory 140 may have stored at least one program that is to be executed by the processor 150. Furthermore, the memory 140 may store data (for example, raw data obtained by scanning an oral cavity, or the like) received from the intraoral scanner 10. Alternatively, the memory 140 may store an oral cavity image 3-dimensionally representing an oral cavity. According to an embodiment, the memory 1400 may include one or more instructions for generating drain holes in teeth model data. According to an embodiment, the memory 1400 may include one or more instructions for performing the method disclosed herein to generate drain holes in teeth model data.

The processor 150 may execute the at least one instruction stored in the memory 140 to control an intended operation to be performed. Here, the at least one instruction may be stored in an internal memory in the processor 150 or may be stored in the memory 140 in a data processing device separately from the processor 150.

Specifically, the processor 150 may execute the at least one instruction to control at least one component in the data processing device such that an intended operation is performed. Therefore, even when descriptions are made by taking an example in which a processor performs certain operations, this may mean that the processor control at least one component of the data processing device such that certain operations are performed.

The processor 150 according to an embodiment may execute the at least one instruction stored in the memory 140 to generate scan data based on raw data obtained by scanning the inside of an oral cavity including teeth or scanning a teeth model. Alternatively, the processor 150 may execute the at least one instruction stored in the memory 140 to obtain the scan data pre-stored in memory or obtain the scan data from an external device.

The processor 150 may execute the at least one instruction stored in the memory 140 to generate a base based on the scan data. The processor 150 may execute the at least one instruction stored in the memory 140 to, in a base creation mode, receive an input for drain hole generation and, when the drain hole generation input is received, provide menus for setting information regarding drain holes.

When the information regarding drain holes, including the number of drain holes, diameters of the drain holes, distances from a base to the drain holes, and the like, is set, the processor 150 may execute the at least one instruction stored in the memory 140 to generate cylinders corresponding to one or more drain holes.

For example, the processor 150 may determine positions of the cylinders, based on the number of drain holes and the distances from the base to the drain holes. In addition, the processor 150 may determine the heights of side surfaces of the cylinders such that the one or more cylinders pass through a wall of the base.

When a user input for moving a first cylinder from among the one or more cylinders generated through the user interface 120 is received, the processor 150 may move the first cylinder along a reference curve of the base.

The processor 150 may execute the at least one instruction stored in the memory 140 to move the first cylinder on the base by converting the user input for selecting and moving the first cylinder into a movement on the base. For example, when a user input for moving the first cylinder by as much as a first distance in a first direction, on 2-dimensional coordinates, is received, the processor 150 may move the first cylinder by as much as a second distance in a second direction along the reference curve of the base.

Because the method, performed by the processor 150, of converting a movement corresponding to the user input, on 2-dimensional coordinates, into a movement on the base, according to an embodiment, has been described in detail with reference to FIG. 7, repeated descriptions are omitted.

According to an embodiment, the processor 150 may execute the at least one instruction stored in the memory 140 to generate one or more drain holes by deleting data corresponding to the one or more cylinders from teeth model data. Here, the processor 150 may distinguish between a tooth region and a gingiva region in the scan data and may control drain holes not to be generated in the tooth region. For example, when one or more cylinders overlap the tooth region, the processor 150 may not delete data corresponding to the overlapping cylinders. Alternatively, when one or more cylinders overlap the tooth region, the processor 150 may adjust positions of the overlapping cylinders and thus place the cylinders in the gingiva region or the base not to overlap the tooth region.

According to an embodiment, the processor 150 may execute the at least one instruction stored in the memory 140 to display the teeth model data in which the drain holes are generated.

According to an embodiment, the processor 150 may be implemented to include, therein, at least one internal processor and a memory device (for example, random access memory (RAM), read-only memory (ROM), or the like) for storing at least one of a program, an instruction, a signal, and data that are to be processed or used by the internal processor.

In addition, the processor 150 may include a graphics processing unit for processing graphics corresponding to a video. In addition, a processor may be implemented by a system-on-chip (SoC) in which a core is integrated with a GPU. Furthermore, the processor may include a single core or multiple cores. For example, the processor may include dual-cores, triple-cores, quad-cores, hexa-cores, octa-cores, deca-cores, dodeca-cores, hexadeca-cores, or the like.

In a disclosed embodiment, the processor 150 may generate an oral cavity image based on a 2-dimensional image received from the intraoral scanner 10.

Specifically, the communication interface 110 may receive data obtained by the intraoral scanner 10, for example, raw data obtained by scanning an oral cavity, according to control by the processor 150. In addition, the processor 150 may generate a 3-dimensional oral cavity image, which 3-dimensionally represents an oral cavity, based on the raw data received by the communication interface 110. For example, the intraoral scanner 10 may include an L camera corresponding to a left field of view and an R camera corresponding to a right field of view, to restore a 3-dimensional image according to an optical triangulation method. In addition, the L camera and the R camera of the intraoral scanner may respectively obtain L image data corresponding to the left field of view and R image data corresponding to the right field of view. Next, the intraoral scanner may transmit the raw data including the L image data and the R image data to the communication interface 110 of the oral cavity image processing device 100.

Then, the communication interface 110 may transfer the received raw data to the processor 150, and the processor 150 may generate the oral cavity image 3-dimensionally representing the oral cavity, based on the transferred raw data.

In addition, the processor 150 may directly receive the oral cavity image 3-dimensionally representing the oral cavity from a server, a medical device, or the like that is external thereto, by controlling the communication interface 110. In this case, the processor 150 may obtain the 3-dimensional oral cavity image, instead of generating the 3-dimensional oral cavity image based on the raw data.

According to a disclosed embodiment, performing, by the processor 150, an operation such as “extracting”, “obtaining”, or “generating” may include controlling, by the processor 150, other components to perform the operations set forth above as well as directly performing, by the processor 150, the operations set forth above by executing at least one instruction.

To implement embodiments disclosed herein, the oral cavity image processing device 100 may include only some of the components shown in FIG. 16 or may include more components in addition to the components shown in FIG. 16.

In addition, the oral cavity image processing device 100 may store and execute dedicated software interworking with the intraoral scanner. Here, the dedicated software may be referred to as a dedicated program, a dedicated tool, or a dedicated application. When the oral cavity image processing device 100 operates to interwork with the intraoral scanner 10, the dedicated software stored in the oral cavity image processing device 100 may be connected to the intraoral scanner 10 and receive, in real time, data obtained by oral cavity scanning. For example, there is dedicated software for processing data obtained through oral cavity scanning by i500, which is a product model from Medit Co., Ltd. Specifically, Medit Co., Ltd. produces and distributes “Medit Link” that is software for processing, managing, using, and/or transmitting data obtained by an intraoral scanner (for example, i500). Here, because the “dedicated software” refers to a program, a tool, or an application capable of operating to interwork with an intraoral scanner, the dedicated software may be used in common by various intraoral scanners that have been developed and are being sold by various manufacturers. In addition, the dedicated software described above may be produced and distributed separately from an intraoral scanner for performing oral cavity scanning.

The oral cavity image processing device 100 may store and execute the dedicated software corresponding to the product model, i500. The dedicated software may perform at least one operation for obtaining, processing, storing, and/or transmitting the oral cavity image. Here, the dedicated software may be stored in the processor. In addition, the dedicated software may provide a user interface for use of the data obtained by the intraoral scanner. Here, a user interface screen provided by the dedicated software may include the oral cavity image generated according to a disclosed embodiment.

An oral cavity image processing method according to an embodiment of the present disclosure may be implemented in the form of program instructions capable of being performed by various computer means and may be recorded on a computer-readable medium. In addition, an embodiment of the present disclosure may be a computer-readable storage medium having recorded thereon one or more programs including at least one instruction for performing the oral cavity image processing method.

The computer-readable storage medium may include program instructions, data files, data structures, or the like alone or in combination thereof. Here, examples of the computer-readable storage medium may include magnetic media such as hard disks, floppy disks, and magnetic tape, optical media such as compact disc read-only memory (CD-ROM) and digital versatile discs (DVDs), magneto-optical media such as floptical disks, and hardware devices configured to store and execute program instructions, such as ROM, RAM, and flash memory.

Here, a machine-readable storage medium may be provided in the form of a non-transitory storage medium. Here, the term “non-transitory storage medium” may mean that a storage medium is a tangible device. In addition, the “non-transitory storage medium” may include a buffer in which data is temporarily stored.

According to an embodiment, an oral cavity image processing method according to various embodiments disclosed in the specification may be provided while included in a computer program product. The computer program product may be distributed in the form of a machine-readable storage medium (for example, CD-ROM). Alternatively, the computer program product may be distributed on-line (for example, download or upload), through an application store (for example, Play Store or the like) or directly between two user equipments (for example, smartphones). Specifically, a computer program product according to a disclosed embodiment may include a storage medium having stored thereon a program including at least one instruction to perform an oral cavity image processing method according to a disclosed embodiment.

Heretofore, although embodiments have been described in detail, the scope of the present disclosure is not limited thereto, and any changes and modifications made by those of ordinary skill in the art based on the fundamental concept, which is defined in the following claims, of the present disclosure should be construed as falling within the scope of the present disclosure.

1. An oral cavity image processing method comprising:
obtaining 3-dimensional oral cavity data by scanning an oral cavity including teeth;
generating a base based on the 3-dimensional oral cavity data;
generating, based on a reference curve of the base, one or more cylinders that pass through a wall of the base;
receiving a user input for moving a first cylinder from among the one or more cylinders;
moving the first cylinder along the reference curve of the base based on the user input; and
generating one or more drain holes by deleting data corresponding to the one or more cylinders from teeth model data including the base.

2. The oral cavity image processing method of claim 1, wherein the reference curve of the base comprises at least one of an inner boundary of the base, an outer boundary of the base, and a curve connecting points located in a middle of the base in a thickness direction of the base.

3. The oral cavity image processing method of claim 1, wherein a direction of a central axis of each of the one or more cylinders is parallel to a direction of a normal vector of a point where the central axis intersects the reference curve.

4. The oral cavity image processing method of claim 1, wherein the moving of the first cylinder along the reference curve of the base comprises:
generating a virtual ray in a direction perpendicular to a screen from a first point on the screen, the first point corresponding to the user input;
determining a second point on the reference curve, the second point being at a minimum distance from the virtual ray; and
moving the first cylinder to locate the second point on the central axis of the first cylinder.

5. The oral cavity image processing method of claim 4, wherein the determining of the second point comprises determining the second point on the reference curve to be at a minimum distance from the virtual ray and within a preset distance from a third point on the reference curve, the third point being an intersection point between the reference curve and the central axis of the first cylinder before the first cylinder is moved.

6. The oral cavity image processing method of claim 1, wherein the moving of the first cylinder along the reference curve of the base comprises moving the first cylinder such that a distance between the central axis of the first cylinder and a reference surface of the base is maintained.

7. The oral cavity image processing method of claim 1, further comprising displaying the teeth model data in which the one or more drain holes are generated.

8. The oral cavity image processing method of claim 7, further comprising obtaining the teeth model data by generating mesh data extending from an edge of the 3-dimensional oral cavity data to the base.

9. The oral cavity image processing method of claim 1, wherein the generating of the one or more cylinders comprises receiving a user input regarding a number of the one or more cylinders, diameters of the one or more cylinders, and distances from a reference surface of the base to the one or more cylinders.

10. The oral cavity image processing method of claim 9, wherein the generating of the one or more cylinders further comprises determining positions of the one or more cylinders, based on the number of the one or more cylinders and the distances from the reference surface of the base to the one or more cylinders.

11. The oral cavity image processing method of claim 1, wherein the generating of the one or more cylinders comprises:
determining heights of side surfaces of the one or more cylinders such that the one or more cylinders pass through the wall of the base; and
generating the one or more cylinders based on the determined heights of the side surfaces of the one or more cylinders.

12. An oral cavity image processing device comprising:
a display;
a user interface;
a memory storing one or more instructions; and
a processor,
wherein the processor is configured to execute the one or more instructions stored in the memory to:
obtain 3-dimensional oral cavity data by scanning an oral cavity including teeth;
generate a base based on the 3-dimensional oral cavity data;
generate, based on a reference curve of the base, one or more cylinders that pass through a wall of the base;
control the display to display the generate one or more cylinders;
receive, through the user interface, a user input for moving a first cylinder from among the one or more cylinders;
move the first cylinder along the reference curve of the base based on the user input; and
generate one or more drain holes by deleting data corresponding to the one or more cylinders from teeth model data including the base.

13. The oral cavity image processing device of claim 12, wherein the reference curve of the base comprises at least one of an inner boundary of the base, an outer boundary of the base, and a curve connecting points located in the middle of the base in a thickness direction of the base.

14. The oral cavity image processing device of claim 12, wherein a direction of a central axis of each of the one or more cylinders is parallel to a direction of a normal vector of a point where the central axis intersects the reference curve.

15. The oral cavity image processing device of claim 12, wherein the processor is further configured to execute the one or more instructions stored in the memory to:
generate a virtual ray in a direction perpendicular to a screen from a first point on the screen, the first point corresponding to the user input;
determine a second point on the reference curve, the second point being at a minimum distance from the virtual ray; and
move the first cylinder to locate the second point on the central axis of the first cylinder.

16. The oral cavity image processing device of claim 15, wherein the processor is further configured to execute the one or more instructions stored in the memory to determine the second point on the reference curve to be at a minimum distance from the virtual ray and within a preset distance from a third point on the reference curve, the third point being an intersection point between the reference curve and the central axis of the first cylinder before the first cylinder is moved.

17. The oral cavity image processing device of claim 12, wherein the processor is further configured to execute the one or more instructions stored in the memory to move the first cylinder such that a distance between the central axis of the first cylinder and a reference surface of the base is maintained.

18. The oral cavity image processing device of claim 12, wherein the processor is further configured to execute the one or more instructions stored in the memory to control the display to display the teeth model data in which the one or more drain holes are generated.

19. The oral cavity image processing device of claim 18, wherein the processor is further configured to execute the one or more instructions stored in the memory to obtain the teeth model data by generating mesh data extending from an edge of the 3-dimensional oral cavity data to the base.

20. A computer-readable recording medium having recorded thereon a program comprising at least one instruction for performing, by a computer, an oral cavity image processing method, the oral cavity image processing method comprising:
obtaining 3-dimensional oral cavity data by scanning an oral cavity including teeth;
generating a base based on the 3-dimensional oral cavity data;
generating, based on a reference curve of the base, one or more cylinders that pass through a wall of the base;
receiving a user input for moving a first cylinder from among the one or more cylinders;
moving the first cylinder along the reference curve of the base based on the user input; and
generating one or more drain holes by deleting data corresponding to the one or more cylinders from teeth model data including the base.
